(19) United States
(12) Patent Application Publication
(10) Pub. No.: US 2012/0239089 A1
(43) Pub. Date: Sep. 20, 2012

(54) INTERSPINOUS PROCESS IMPLANT AND METHOD OF IMPLANTATION

(75) Inventors: Calin Druma, San Jose, CA (US); Bruce Chabansky, Palo Alto, CA (US)

(73) Assignee: Kyphon SARL, Neuchatel (CH)

(21) Appl. No.: 13/050,183

(22) Filed: Mar. 17, 2011

(51) Int. Cl. A61B 17/70

(52) U.S. Cl. 606/249

(57) ABSTRACT

Medical devices for the treatment of spinal conditions are described herein. The medical device includes a sleeve, and optionally a bumper, that is disposed between adjacent spinous processes and has a first retention member and a second retention member, which may be locked together.

### BACKGROUND

This invention relates generally to devices for the treatment of spinal conditions, and more particularly, to the treatment of various spinal conditions that cause back pain. Even more particularly, this invention relates to devices that may be placed between adjacent spinous processes to treat various spinal conditions. For example, spinal conditions that may be treated with these devices may include spinal stenosis, degenerative disc disease (DDD), disc herniations and spinal instability, among others.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal, cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis and other spinal conditions. The principal goal of surgery to treat lumbar spinal stenosis is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia and an incision is made in the patient to access the spine. The lamina of one or more vertebrae may be partially or completely removed to create more space for the nerves. The success rate of decompressive laminectomy has been reported to be in excess of 65%. A significant reduction of the symptoms of lumbar spinal stenosis is also achieved in many of these cases.

The failures associated with a decompressive laminectomy may be related to postoperative iatrogenic spinal instability. To limit the effect of iatrogenic instability, fixation and fusion may also be performed in association with the decompression. In such a case, the intervertebral disc may be removed, and the adjacent vertebrae may be fused. A discectomy may also be performed to treat DDD and disc herniations. In such a case, a spinal fusion would be required to treat the resulting vertebral instability. Spinal fusion is also traditionally accepted as the standard surgical treatment for lumbar instability. However, spinal fusion sacrifices normal spinal motion and may result in increased surgical complications. It is also believed that fusion to treat various spinal conditions may increase the biomechanical stresses imposed on the adjacent segments. The resultant altered kinematics at the adjacent segments may lead to accelerated degeneration of these segments.

As an alternative or complement to the surgical treatments described above, an interspinous process device may be implanted between adjacent spinous processes of adjacent vertebrae. The purposes of these devices are to provide stabilization after decompression, to restore foraminal height, and to unload the facet joints. They also allow for the preservation of a range of motion in the adjacent vertebral segments, thus avoiding or limiting possible overloading and early degeneration of the adjacent segments as induced by fusion. The vertebrae may or may not be distracted before the device is implanted therebetween. An example of such a device is the interspinous prosthesis described in U.S. Pat. No. 6,626,944, the entire contents of which are expressly incorporated herein by reference in its entirety. This device, commercially known as the DIAM® spinal stabilization system, is designed to restabilize the vertebral segments as a result of various surgical procedures or as a treatment of various spinal conditions. It limits extension and may act as a shock absorber, since it provides compressibility between the adjacent vertebrae, to decrease intradiscal pressure and reduce abnormal segmental motion and alignment. This device provides stability in all directions and maintains the desired separation between the vertebral segments all while allowing motion in the treated segment.

Although currently available interspinous process devices typically work for their intended purposes, they could be improved. For example, where the spacer portion of the implant is formed from a hard material to maintain distraction between adjacent vertebrae, point loading of the spinous process can occur due to the high concentration of stresses at the point where the hard material of the spacer contacts the spinous process. This may result in excessive subsidence of the spacer into the spinous process. In addition, if the spinous process is osteoporotic, there is a risk that the spinous process could fracture when the spine is in extension.

In addition, because of the human anatomy and the complex biomechanics of the spine, some currently available interspinous process devices may not be easily implantable. The spine is divided into regions that include the cervical, thoracic, lumbar, and sacrococcygeal regions. The cervical region includes the top seven vertebrae indentified as C1-C7. The thoracic region includes the next twelve vertebrae identified as T1-T12. The lumbar region includes five vertebrae L1-L5. The sacrococcygeal region includes five fused vertebrae comprising the sacrum. These five fused vertebrae are identified as the S1-S5 vertebrae. Four or five rudimentary members form the coccyx. The interspinous ligament connects adjacent spinous processes and extends between the spinous processes. The supraspinous ligament is a very strong band connecting the contiguous spinous processes of the vertebrae of the spine and is located along the posterior ends of the spinous processes. It extends from C7 to the sacrum. Some interspinous process devices require that the supraspinous ligament be cut in the area between adjacent spinous processes that define the interspinous space into which the interspinous process device is to be implanted. This is because the configuration of such implants makes it difficult to insert the implant laterally into the interspinous space because the spinous processes limit the vertical height of the device that can be implanted laterally into the interspinous space. For these implants, the supraspinous ligament may have to be cut to allow the implant to be positioned in the interspinous space through a posterior to anterior implantation method. It is possible that cutting the supraspinous ligament may compromise the adjacent segment kinematics.

Thus, a need exists for an interspinous process device that may be readily positioned in a patient's anatomy. Moreover, there is a need to provide an interspinous process device that can provide dynamic stabilization to the instrumented motion segment and not affect adjacent segment kinematics.

### SUMMARY OF THE INVENTION

The interspinous process device of this invention includes (i) a first retention member having a sleeve that is adapted to be disposed between adjacent spinous processes wherein the first retention member is adapted to be disposed along a lateral side of a superior spinous process, and an inferior spinous process, and (ii) a second retention member adapted to be connected to the first retention member wherein the second retention member is disposed along an opposite lateral side of the superior spinous process and the inferior spinous process. A bumper may also be disposed about the sleeve. For ease of reference, the term “shaft” when used hereinafter refers to embodiments that include only the sleeve and that include both the sleeve and the bumper. The length of the major axes of the first retention member and the second retention member is greater than the distance between adjacent spinous processes when they are distracted to the desired spacing. A suitable locking mechanism is provided to lock the first retention member to the second retention member. The locking mechanism ensures that when the first retention member and the second retention member are properly located in the patient's anatomy and are connected together, the major axes of the first retention member and the second retention member extend in a direction that is aligned and parallel to each other with the major axes of the first and second retention members extending in a direction that is generally parallel to the sagittal and coronal planes and generally normal to the axial plane.

With the device of this invention located in place in the patient's anatomy, the shaft is disposed between the adjacent spinous processes and is substantially perpendicular to, and crosses through, the sagittal plane. The diameter of the shaft should be sufficient to provide the desired distraction between the adjacent spinous processes to achieve the expected therapeutic outcome from implantation of the device. It is to be understood that the shaft may be located on the second retention member if desired. In addition, the sleeve may be formed so it is hollow and may extend from either the first retention member or the second retention member. A solid or hollow core may be formed on the other of the second retention member or the first retention member. The solid or hollow core fits inside the sleeve, with the locking mechanism formed on the core and the sleeve. The sleeve and/or bumper may be formed from a softer, more flexible, compressible or compliant material and the core may be formed from a harder, more rigid material. For example, the sleeve and/or bumper may be formed from silicone and the core may be formed from titanium or PEEK. The sleeve may also be formed from titanium or PEEK if a soft bumper is used. Whether the sleeve or the core is formed from a harder material, the shaft acts as an extension stop when the spine moves in extension to maintain the desired distraction between adjacent spinous processes.

The method of implanting the interspinous process device avoids the need to create a large medial line incision in the patient or the dissection of soft tissue at the affected level. Instead, the interspinous process device may be implanted in a minimally invasive manner. The shaft of the first retention member is inserted through the interspinous ligament, which has been dissected to create an opening therethrough, from one side of the adjacent spinous processes. Dissecting the interspinous ligament allows passage of the shaft of the first retention member therethrough, and through the space between adjacent spinous processes with a lateral approach. The first retention member is oriented such that the major axis of the first retention member is generally parallel to the sagittal and coronal planes and is generally normal to the axial plane. Similarly, the core of the second retention member may be inserted through the interspinous ligament from the other side of the adjacent spinous processes. The second retention member is oriented such that its major axis extends in a direction that is generally parallel to the major axis of the first retention member and the sagittal and coronal planes and is generally normal to the axial plane. The core is inserted into the lumen of the sleeve and the locking mechanism locks the sleeve and the core, and thus the first and second retention members, together. As noted above, the major axes of the first retention member and the second retention member define a dimension that is greater than the distance between adjacent spinous processes. In this manner, the interspinous process device is held in place by the first and second retention members and the shaft prevents the space between the adjacent spinous processes from collapsing during extension of the spine.

### BRIEF DESCRIPTION OF THE DRAWINGS

FIG. 1 is a perspective view of an interspinous process device mounted in a spine;

FIG. 2 is a perspective view of an interspinous process device;

FIG. 3 is a front elevation view of the separate elements of the interspinous process device shown in FIG. 2;

FIG. 4 is a front elevation view of the interspinous process device shown in FIG. 2; and

FIG. 5 is a cross-sectional view of an interspinous process device taken along lines V-V of FIG. 2.

### DETAILED DESCRIPTION

As used in this specification and the appended claims, the singular forms “a,” “an” and “the” include plural referents unless the context clearly dictates otherwise. Thus, for example, the term “a member” is intended to mean a single member or a-combination of members, and “a material” is intended to mean one or more materials, or a combination thereof. Typically, the words “proximal” and “distal” refer to directions or locations closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device. However, for the device described herein, “distal” refers to a location toward the left in the FIGS. and “proximal” refers to a location toward the right in the FIGS. This convention is used herein to avoid confusion since each element of the device described herein may be inserted from opposite sides of the spine.

As used in this specification and the appended claims, the terms “upper”, “top”, “lower”, “bottom”, “front”, “back”, “rear”, “left”, “right”, “side”, “middle” and “center” refer to portions of or positions on the implant when the implant is oriented in its implanted position.

As used in this specification and the appended claims, the term “axial plane” when used in connection with particular relationships between various parts of the implant means a plane that divides the implant into upper and lower parts. As used in this specification and the appended claims, the term “coronal plane” when used in connection with particular relationships between various parts of the implant means a plane that divides the implant into front and back parts. As used in this specification and the appended claims, the term “sagittal plane” when used in connection with particular relationships between various parts of the implant means a plane that divides the implant into left and right parts.

As used in this specification and the appended claims, the term “body” when used in connection with the location where the device of this invention is to be placed to treat spinal disorders, or to teach or practice implantation methods for the device, means a mammalian body. For example, a body can be a patient's body, or a cadaver, or a portion of a patient's body or a portion of a cadaver or a model of any of the foregoing.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, “perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

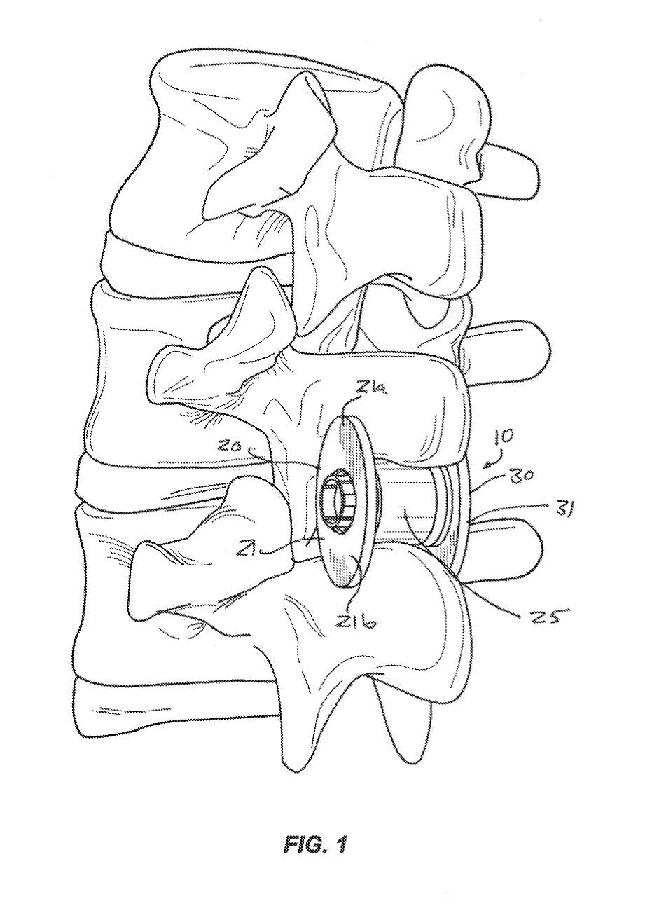

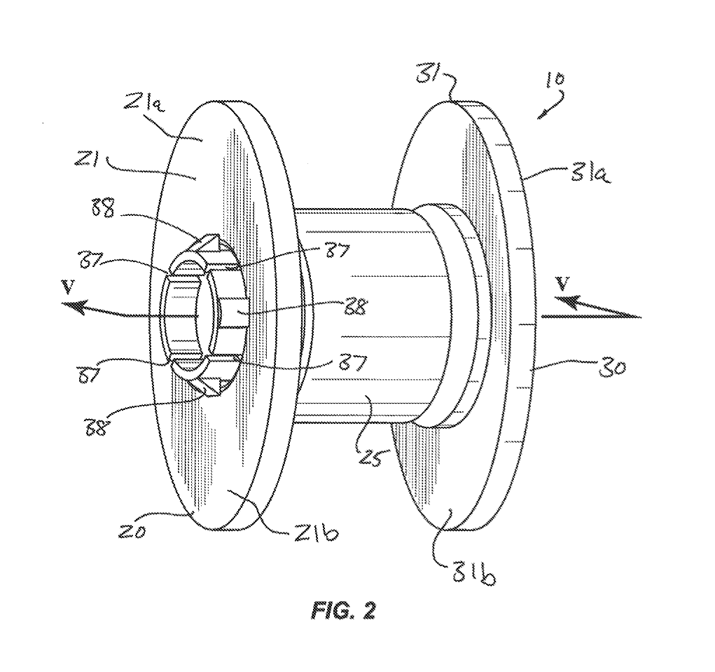

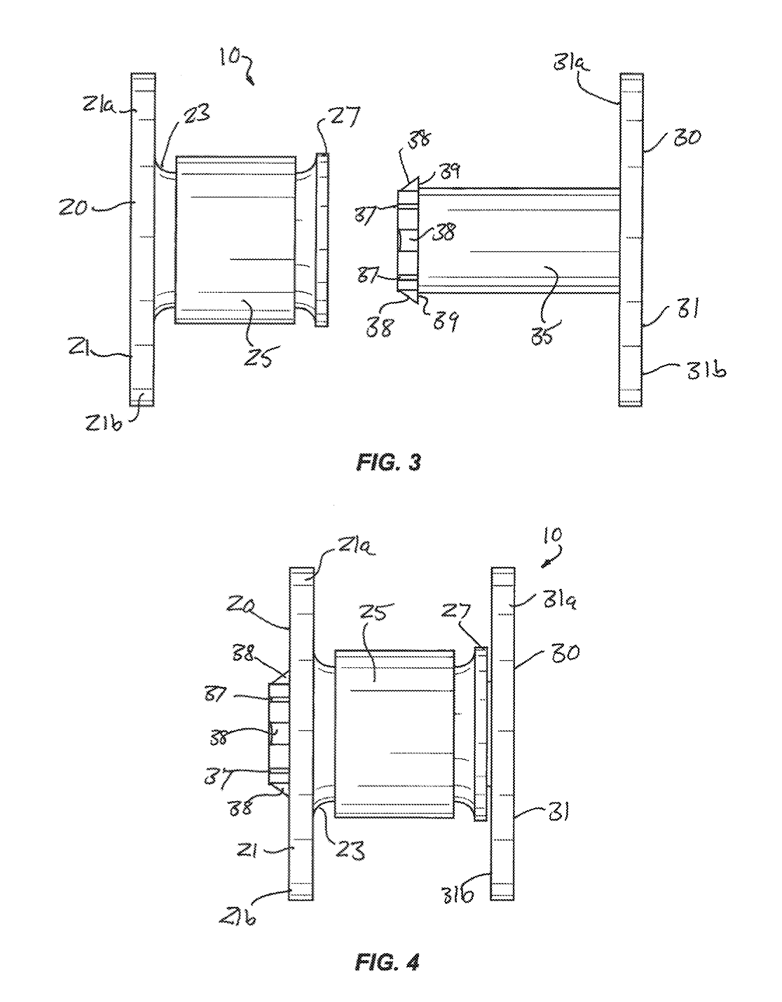

The interspinous process device 10 of this invention includes a first retention member 20 having a sleeve 23 extending from a medial portion thereof, and a second retention member 30 having a core 35 extending from a medial portion thereof. Sleeve 23 may be hollow. A bumper 25 may also be disposed about sleeve 23, which may include a proximal flange 27 to prevent bumper 25 from sliding off of sleeve 23. Bumper 25 may have a length substantially the same as the length of sleeve 23 or it may be somewhat shorter. However, the length of bumper 25, and indeed sleeve 23, should be sufficient to extend across the width of a typical spinous process with some room to spare. See FIG. 1. Bumper 25 may be free to rotate around sleeve 23 to facilitate posterior and anterior movement of device 10 during implantation. Alternatively, bumper 25 may be fixed with respect to sleeve 23. It is to be understood that sleeve 23 need not include bumper 25, but the following description will describe device 10 as including bumper 25. Bumper 25 is adapted to be disposed between adjacent spinous processes with first retention member 20 adapted to be disposed along a lateral side of an adjacent superior spinous process and inferior spinous process.

As shown in FIG. 1, first retention member 20 is adapted to be disposed along the left lateral sides of the superior and inferior spinous processes. Core 35 is adapted to be disposed inside sleeve 23 with second retention member 30 adapted to be disposed along the opposite lateral sides of the adjacent superior and inferior spinous processes. It is to be understood that sleeve 23 could extend from the medial portion of second retention member 30 and core 35 could extend from the medial portion of first retention member 20. However, for ease of description, sleeve 23 will be described herein as extending from first retention member 20 and core 35 will be described herein as extending from second retention member 30.

First retention member 20 includes a first wing 21 having a first superior portion 21a and a first inferior portion 21b, with sleeve 23 extending from a medial portion of the proximal face of first retention member 20. As shown in FIG. 1, first superior portion 21a is adapted to engage the left lateral side of a superior spinous process when device 10 is appropriately located in the space between adjacent spinous processes such that the longitudinal axis of sleeve 23 is generally perpendicular to the sagittal plane. In this position, first inferior portion 21b is adapted to engage the left lateral side of the adjacent inferior spinous process. As shown herein, first wing 21 has a generally elliptical configuration with a major axis and a minor axis. It is to be understood that any other geometrical shape may be used for first wing 21. However, no matter what the geometric shape is, the dimension of first retention member 20 along the major axis should be greater than the distance between adjacent spinous processes when they are distracted to the desired spacing.

Second retention member 30 includes a second wing 31 having a second superior portion 31a and a second inferior portion 31b with core element 35 extending from a medial portion of the distal face of second wing 31. Core 35 may be hollow or solid, depending on the flexibility characteristics desired for the shaft. As shown herein, second retention member 30 has a generally elliptical configuration with a major axis and a minor axis. Although second retention member 30 may be formed as an elliptical configuration, any other geometrical shape may be used from second retention member 30. However, the dimension of second retention member 30 along the major axis should be greater than the distance between adjacent spinous processes when they are distracted to the desired spacing.

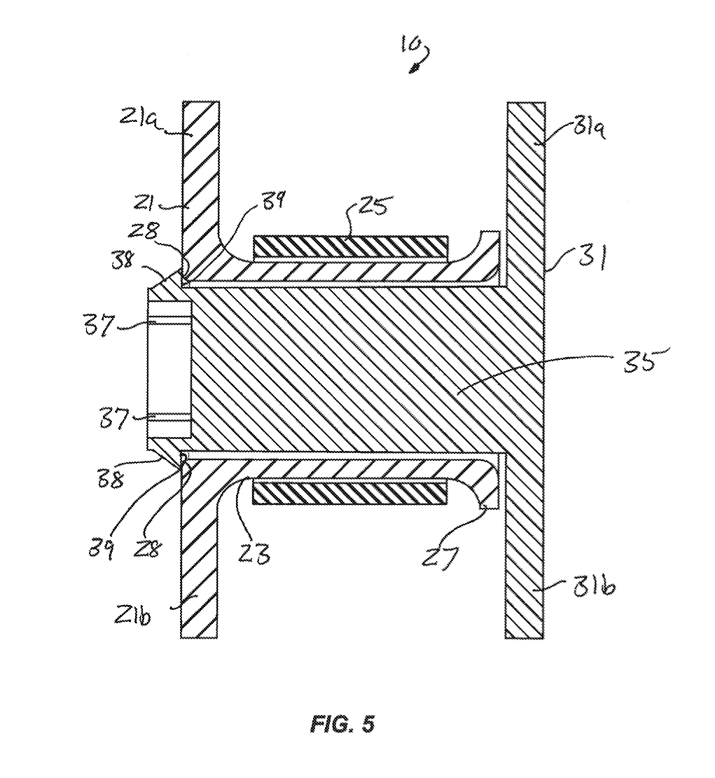

A plurality of lugs 38 may be spaced around the periphery of the distal end of core 30. Lugs 38 may have a generally tapered configuration with a smaller distal end and a larger proximal end, with the proximal end defining a proximal face 39 of the lug. The smaller distal end of lugs 38 allows core 35 to be inserted in the proximal end of sleeve 23 and moved distally through the lumen of sleeve 23. Lugs 38 may be formed on the distal end of core 35. In one configuration, the distal end of core 35 is formed as an annular ring defined by a relatively thin circumferential strip of material extending from the distal end of a solid core body. This configuration provides lugs 38 with some ability to flex inwardly when sleeve 23 forces lugs 38 inwardly and thus allows core 35 to slide through the lumen of sleeve 23 with minimal resistance. In addition, the flexibility allows lugs 38 to spring back to their original position when the inward force is released. For more flexibility, the annular ring may define a plurality of slots 37 on either side of each of lugs 38 to ensure that lugs 38 may flex inwardly with little resistance. Slots 37 may be arranged symmetrically about the annular ring between and adjacent to each lug 38. Once the distal end of core 35, and thus lugs 38, extend past the distal end of first wing 21 so lugs 38 are adjacent to the distal face of first wing 21, lugs 38 spring back to their original position. In this position, each proximal face 39 of each lug 38 engages the distal inner edge 28 of first wing 21. See e.g. FIG. 5. This engagement between each proximal face 39 of each lug 38 and distal inner edge 28 of first wing 21 locks first retention member 20 to second retention member 30. Of course, the dimensions of core 35 and sleeve 23 must be defined so as to allow this desired engagement between proximal faces 39 and distal inner edge 28. Preferably, four lugs 38 may be used and may be spaced around the circumference of the distal end of core 30. Lugs 38 may be spaced 90 degrees apart or at some other distance apart. It is to be understood than any number of lugs may be used as long as the number used is sufficient to connect first retention member 20 to second retention member 30.

The interspinous ligament is typically dissected with a cutting instrument, such as a simple scalpel, an electrosurgical device or the like, not shown, to create an appropriately sized opening in the interspinous ligament to allow passage of the shaft of first retention member therethrough. This allows device 10 to be implanted in the space between adjacent spinous processes with a lateral approach. In some circumstances, the space between adjacent spinous processes may first need to be distracted with a distraction tool, not shown, to provide additional space and pain relief for the patient. After the physician confirms sufficient distraction, device 10 can then be placed in the space between the adjacent spinous processes. Device 10 can come in different sizes to accommodate different amounts of distraction/space needed between adjacent spinous processes.

Sleeve 23 and bumper 25 of first retention member 20 are inserted through the opening formed in the interspinous ligament from the distal side of the adjacent spinous processes. First retention member 20 is oriented such that its major axis is generally parallel to the sagittal and coronal planes. First retention member 20 is moved proximally sufficiently so that sleeve 23 and bumper 25 are located between adjacent superior and inferior spinous processes and extend across the sagittal plane. Importantly, the supraspinous ligament remains undisturbed during the procedure. Core 35 of second retention member 30 is inserted into the lumen of sleeve 23 from the proximal side of the adjacent spinous processes such that second retention member 30 extends along the proximal side of adjacent superior and inferior spinous processes with the major axis generally parallel to the sagittal and coronal planes. First retention member 20 and second retention member 30 are locked together by the engagement of each proximal face 39 of each lug 38 with distal inner edge 28 of first wing 21. Either first retention member 20 or second retention member 30 may be inserted into the intraspinous space first, or they may be inserted in that space substantially simultaneously. The major axes of first retention member 20 and second retention member 30 are oriented so they extend in the same direction and are generally parallel to each other and the sagittal and coronal planes. As noted above, the major axes of first retention member 20 and second retention member 30 respectively define a dimension that is greater than the distance between adjacent spinous processes. Of course, the distance between first retention member 20 and second retention member 30 should be slightly greater than the distance between the distal side of the adjacent spinous process and the proximal side of the adjacent spinous processes. In this manner, device 10 is held in place by first retention member 20 and second retention member 30.

Device 10 can be constructed with various biocompatible materials such as, for example, titanium, titanium alloy, surgical steel, biocompatible metal alloys, stainless steel, Nitinol, plastic, polyetheretherketone (PEEK), carbon fiber, ultra-high molecular weight (UHMW) polyethylene, and other biocompatible polymeric materials. The material of device 10 may have, for example, a compressive strength similar to or higher than that of bone. Bumper 25 may be formed from a material having an elastic modulus higher than the elastic modulus of the bone of the spinous processes. In another embodiment, bumper 25 is not used and sleeve 23 may be formed from a material having a higher elastic modulus than the materials used to form the remainder of first retention member 20 and second retention member 30. For example, sleeve 23 may have an elastic modulus higher than bone, while the remainder of first retention member 20 and second retention member 30, including core 35, may have a lower elastic modulus than bone. Bumper 25 and/or sleeve 23 may be formed of a compliant material, such as silicone, to dampen the shock when the spinal column is moved into extension.

While various embodiments of the device have been described above, it should be understood that they have been presented by way of example only, and not limitation. The foregoing description of the interspinous process device is not intended to be exhaustive or to limit the invention of the device. Many modifications and variations will be apparent to the practitioner skilled in the art. It is intended that the scope of the invention be defined by the following claims and their equivalents.

What is claimed is:

1. A device, comprising:
a first retention member having a proximal face and a distal face and a sleeve extending from a medial portion of the proximal face, the distal face defining a distal inner edge adjacent to the medial portion;
a second retention member having a core extending from a medial portion thereof wherein the core is adapted to be disposed in the outer sleeve; and
at least one lug disposed on an end of the sleeve wherein the lug is adapted to engage the distal inner edge.

2. The device of claim 1 further comprising a tubular bumper disposed about the sleeve.

3. The device of claim 2 wherein the bumper is formed from a resiliently compressible material.

4. The device of claim 3 wherein the bumper is formed from silicone.

5. The device of claim 1 wherein the sleeve is formed from a resiliently compressible material.

6. The device of claim 5 wherein the sleeve is formed from silicone.

7. The device of claim 1 wherein the core is formed from a material more rigid than a material forming the sleeve.

8. The device of claim 7 wherein the core is formed from titanium.

9. The device of claim 2 wherein the core is formed from a material more rigid than a material forming the bumper.

10. A method of implanting a medical device into an interspinous space defined between a superior spinous process and an inferior spinous process, comprising:
providing a first retention member having a sleeve;
inserting the sleeve through an interspinous space from a distal side of the superior spinous process and the inferior spinous process so that the first retention member is adjacent a distal side of the superior spinous process and a distal side of the inferior spinous process;
providing a second retention member having a core element;
inserting the core element into the sleeve from a proximal side of the superior spinous process and a proximal side of the inferior spinous process; and
locking the distal retention member with respect to the proximal retention member wherein a major axis of the proximal retention member and a major axis of the distal retention member extend in directions that are substantially parallel to each other and to a sagittal plane and a coronal plane.

11. The method of claim 10 wherein the first retention member and the second retention member are inserted into the intraspinous space substantially simultaneously.

12. The method of claim 10 wherein the first retention member is inserted into the intraspinous space before the second retention member.

13. The method of claim 10 wherein the first retention member is inserted into the intraspinous space after the second retention member.

14. The method of claim 10 further comprising the step of enlarging the intraspinous space prior to inserting the first retention member.

15. The method of claim 10 wherein the device further comprises a bumper rotatably disposed about the sleeve and further comprising the step of moving the device in a posterior to anterior or anterior to posterior direction during implanting.

16. A device, comprising:
a first retention member having a first proximal face and a first distal face and a sleeve extending from a first medial portion of the first proximal face, the first distal face defining a distal inner edge adjacent to the first medial portion;
a second retention member having a second proximal face and a second distal face and a core extending from a second medial portion thereof wherein the core is adapted to be disposed in the outer sleeve and the core includes an annular distal portion; and
a plurality of lugs disposed about the annular distal portion of the core, wherein the lugs are adapted to engage the distal inner edge.

17. The device of claim 16 wherein the plurality of lugs is four lugs disposed about 90 degrees apart around the annular distal portion of the core.

18. The device of claim 17 further comprising a bumper disposed about the sleeve.

19. The device of claim 16 wherein the core includes a solid proximal and medial portion.

20. The device of claim 16 wherein the annular distal portion defines a plurality of slots such that each slot of the plurality of slots is disposed between each lug of the plurality of lugs.
